(19) United States
(12) Patent Application Publication
(10) Pub. No.: US 2024/0011938 A1
(43) Pub. Date: Jan. 11, 2024

(54) SENSOR ELEMENT AND GAS SENSOR

(71) Applicant: NGK INSULATORS, LTD., Nagoya, Aichi (JP)

(72) Inventors: Takayuki SEKIYA, Nisshin (JP); Yusuke WATANABE, Nagoya (JP); Kodai ICHIKAWA, Nagoya (JP)

(21) Appl. No.: 18/472,569

(22) Filed: Sep. 22, 2023

Related U.S. Application Data
(63) Continuation of application No. PCT/JP2022/014340, filed on Mar. 25, 2022.

(30) Foreign Application Priority Data
Mar. 31, 2021 (JP) 2021-059122

(51) Int. Cl. G01N 27/409; G01N 33/00; G01N 27/41

(52) U.S. Cl. CPC G01N 27/409; G01N 33/0027; G01N 27/41

(57) ABSTRACT

A sensor element is for detecting a specific gas concentration in a measurement-object gas, and includes: an element body internally provided with a measurement-object gas flow portion; an adjustment chamber pump cell having an adjustment electrode disposed in an oxygen concentration adjustment chamber of the measurement-object gas flow portion, and a pump outer electrode disposed outside the element body, the adjustment chamber pump cell being configured to pump out oxygen from the oxygen concentration adjustment chamber or pump oxygen into the oxygen concentration adjustment chamber; a reference-gas introduction portion disposed inside the element body; and an outer sensor cell having a voltage outer electrode disposed outside the element body, and a reference electrode disposed inside the element body, the outer sensor cell being configured to generate a voltage based on an oxygen concentration in the measurement-object gas outside the element body.

### CROSS-REFERENCE TO RELATED APPLICATION

This application is a continuation application of PCT/JP2022/014340, filed on Mar. 25, 2022, which claims the benefit of priority of Japanese Patent Application No. 2021-059122, filed on Mar. 31, 2021, the entire contents of which are incorporated herein by reference.

### BACKGROUND OF THE INVENTION

### 1. Field of the Invention

The present invention relates to a sensor element and a gas sensor.

### 2. Description of the Related Art

Hitherto, a known gas sensor detects the concentration of a specific gas, such as NOx, in a measurement-object gas, such as the exhaust gas of an automobile. For example, Patent Literature 1 describes a gas sensor including an elongate plate-shaped sensor element obtained by stacking a plurality of oxygen-ion-conductive solid electrolyte layers.

A schematic cross-sectional view schematically illustrating an example of the configuration of a gas sensor 900 in such a related art is illustrated in FIG. 9. As illustrated, the gas sensor 900 includes a sensor element 901. The sensor element 901 is an element having a structure in which oxygen-ion-conductive solid electrolyte layers 911 to 916 are stacked. In the sensor element 901, a measurement-object gas flow portion that introduces a measurement-object gas is formed between the lower surface of the solid electrolyte layer 916 and the upper surface of the solid electrolyte layer 914, and the measurement-object gas flow portion is provided with a first internal cavity 920, a second internal cavity 940, and a third internal cavity 961. An inner pump electrode 922 is disposed in the first internal cavity 920, an auxiliary pump electrode 951 is disposed in the second internal cavity 940, and a measurement electrode 944 is disposed in the third internal cavity 961. In addition, an outer pump electrode 923 is disposed on the upper surface of the solid electrolyte layer 916. In contrast, between the upper surface of the solid electrolyte layer 913 and the lower surface of the solid electrolyte layer 914, a reference electrode 942 is disposed which is in contact with a reference gas (e.g., atmospheric gas) serving as a reference for detecting a specific gas concentration in a measurement-object gas. A main pump cell 921 is formed by the inner pump electrode 922, the outer pump electrode 923, and the solid electrolyte layers 914 to 916. A measurement pump cell 941 is formed by the measurement electrode 944, the outer pump electrode 923, and the solid electrolyte layer 914 to 916. A measurement-pump-control oxygen-partial-pressure detection sensor cell 982 is formed by the measurement electrode 944, the reference electrode 942, and the solid electrolyte layers 914, 913. A Vref detection sensor cell 983 is formed by the outer pump electrode 923, the reference electrode 942, and the solid electrolyte layers 913 to 916. A reference-gas adjustment pump cell 990 is formed by the outer pump electrode 923, the reference electrode 942, and the solid electrolyte layers 913 to 916. In the gas sensor 900, when a measurement-object gas is introduced into the measurement-object gas flow portion, oxygen is pumped out or pumped in between the first internal cavity 920 and the outside of the sensor element by the main pump cell 921, and oxygen is further pumped out or pumped in between the second internal cavity 940 and the outside of the sensor element to adjust the oxygen concentration in the measurement-object gas flow portion. NOx in the measurement-object gas after adjustment of the oxygen concentration is reduced in the periphery of the measurement electrode 944. A voltage Vp2 applied to the measurement pump cell 941 is feedback-controlled so that voltage V2 generated in the measurement-pump-control oxygen-partial-pressure detection sensor cell 982 reaches a predetermined target value, thus the measurement pump cell 941 pumps out the oxygen in the periphery of the measurement electrode 944. The NOx concentration in the measurement-object gas is detected based on the pump current Ip2 which flows through the measurement pump cell 941 then. The reference-gas adjustment pump cell 990 pumps oxygen into the periphery of the reference electrode 942 by passing a pump current Ip3 by a voltage Vp3 applied across the reference electrode 942 and the outer pump electrode 923. Thus, when the oxygen concentration of the reference gas in the periphery of the reference electrode 942 decreases, the decrease in the oxygen concentration can be compensated, and reduction in the accuracy of detection of the specific gas concentration is prevented. Furthermore, a voltage Vref is generated between the outer pump electrode 923 and the reference electrode 942 in the Vref detection sensor cell 983. The voltage Vref makes it possible to detect the oxygen concentration in the measurement-object gas outside the sensor element 901.

### CITATION LIST

### Patent Literature

PTL 1: WO 2020/004356 A1

### SUMMARY OF THE INVENTION

Meanwhile, when the oxygen concentration in the measurement-object gas outside the sensor element is detected with the voltage of a sensor cell, such as the voltage Vref of the aforementioned Vref detection sensor cell 983, further improvement of the accuracy of detection of the oxygen concentration has been demanded.

The present invention has been made to solve the aforementioned problem, and a main object thereof is to improve the accuracy of detection of the oxygen concentration in the measurement-object gas using an outer sensor cell.

In order to achieve the aforementioned main object, the present invention employs the following solutions.

A sensor element of the present invention is for detecting a specific gas concentration in a measurement-object gas, and includes: an element body including an oxygen-ion-conductive solid electrolyte layer and internally provided with a measurement-object gas flow portion that introduces a measurement-object gas and causes the measurement-object gas to flow therethrough; an adjustment chamber pump cell having an adjustment electrode disposed in an oxygen concentration adjustment chamber of the measurement-object gas flow portion, and a pump outer electrode disposed outside the element body, the adjustment chamber pump cell being configured to pump out oxygen from the oxygen concentration adjustment chamber or pump oxygen into the oxygen concentration adjustment chamber; a measurement pump cell having a measurement electrode disposed in a measurement chamber provided downstream of the oxygen concentration adjustment chamber of the measurement-object gas flow portion, and the pump outer electrode, the measurement pump cell being configured to pump out oxygen produced from the specific gas in the measurement chamber; a reference-gas introduction portion disposed inside the element body, the reference-gas introduction portion being configured to introduce a reference gas serving as a reference for detecting a specific gas concentration in the measurement-object gas; and an outer sensor cell having a voltage outer electrode disposed outside the element body, and a reference electrode disposed inside the element body so as to be in contact with the reference gas introduced to the reference-gas introduction portion, the outer sensor cell being configured to generate a voltage based on an oxygen concentration in the measurement-object gas outside the element body.

The sensor element includes: an adjustment chamber pump cell that pumps out oxygen from the oxygen concentration adjustment chamber inside the element body or pumps oxygen into the oxygen concentration adjustment chamber; a measurement pump cell that pumps out oxygen from the measurement chamber provided downstream of the oxygen concentration adjustment chamber; and an outer sensor cell that generates a voltage based on an oxygen concentration in the measurement-object gas outside the element body. The pump outer electrode constituting part of the adjustment chamber pump cell and the measurement pump cell, and the voltage outer electrode constituting part of the outer sensor cell are each disposed outside the element body. In other words, in the sensor element, the pump outer electrode and the voltage outer electrode are separately provided outside the element body. Thus, unlike when one electrode serves as the pump outer electrode as well as the voltage outer electrode (e.g., in the sensor element 901 illustrated in FIG. 9, the outer pump electrode 923 serves as the electrodes of the main pump cell 921 and the measurement pump cell 941 as well as the electrode of the Vref detection sensor cell 983), the pump current of the adjustment chamber pump cell and the measurement pump cell does not flow through the voltage outer electrode, thus the voltage of the outer sensor cell does not include a voltage drop of the voltage outer electrode due to the pump current.

Consequently, the voltage of the outer sensor cell has a value which corresponds with higher accuracy to the oxygen concentration in the measurement-object gas outside the element body, thus the accuracy of detection of the oxygen concentration in the measurement-object gas using the outer sensor cell is improved.

A sensor element of the present invention may further include a reference-gas adjustment pump cell having the pump outer electrode and the reference electrode, the reference-gas adjustment pump cell being configured to pump oxygen into a periphery of the reference electrode from a periphery of the pump outer electrode. In this manner, the reference-gas adjustment pump cell pumps oxygen into the periphery of the reference electrode, thus reduction in the oxygen concentration of the reference gas in the periphery of the reference electrode can be supplemented.

In the sensor element of the present invention, the oxygen concentration adjustment chamber may have the first internal cavity provided in the measurement-object gas flow portion, and the second internal cavity provided downstream of the first internal cavity in the measurement-object gas flow portion, the adjustment electrode may have an inner pump electrode disposed in the first internal cavity and an auxiliary pump electrode disposed in the second internal cavity, and the adjustment chamber pump cell may include a main pump cell that has the inner pump electrode and the pump outer electrode and pumps out oxygen from the first internal cavity or pumps oxygen into the first internal cavity, and an auxiliary pump cell that has the auxiliary pump electrode and the pump outer electrode and pumps out oxygen from the second internal cavity or pumps oxygen into the second internal cavity.

A gas sensor of the present invention includes: the sensor element according to any one of the aspects described above; an adjustment chamber pump cell controller that causes the adjustment chamber pump cell to pump out oxygen from the oxygen concentration adjustment chamber or pump oxygen into the oxygen concentration adjustment chamber by controlling the adjustment chamber pump cell so that an oxygen concentration in the oxygen concentration adjustment chamber reaches a predetermined low concentration; and an oxygen concentration detector that detects an oxygen concentration in the measurement-object gas outside the element body based on the voltage of the outer sensor cell.

In the gas sensor, the adjustment chamber pump cell controller controls the adjustment chamber pump cell so that the oxygen concentration in the oxygen concentration adjustment chamber reaches a predetermined low concentration. In this situation, for example, when the oxygen concentration in the measurement-object gas is switched between a high state in which the oxygen concentration is higher than a predetermined low concentration and a low state, the adjustment chamber pump cell controller switches the direction of oxygen moved by the adjustment chamber pump cell to the reverse direction. Thus, the direction of the pump current which flows through the adjustment chamber pump cell is switched to the reverse direction. Therefore, when one electrode serves as the pump outer electrode as well as the voltage outer electrode, the change in the voltage of the outer sensor cell also becomes slow due to the time required for current change when the direction of the pump current flowing through the adjustment chamber pump cell is switched to the reverse direction. In contrast, the gas sensor of the present invention is provided with the pump outer electrode and the voltage outer electrode separately, thus the voltage of the outer sensor cell is not affected by the time required for change in the pump current which flows through the adjustment chamber pump cell, and therefore, the change in the voltage of the outer sensor cell does not become slow. In other words, when the oxygen concentration in the measurement-object gas is switched between a high state in which the oxygen concentration is higher than a predetermined low concentration and a low state, the responsiveness of the voltage is not likely to reduce.

The gas sensor of the present invention may include a reference-gas adjustment unit that causes the reference-gas adjustment pump cell to pump oxygen into the periphery of the reference electrode by applying a control voltage repeatedly turned ON/OFF to the reference-gas adjustment pump cell. In this case, the oxygen concentration detector may obtain the voltage of the outer sensor cell in a period when the repeatedly turned ON/OFF control voltage is OFF.

### BRIEF DESCRIPTION OF THE DRAWINGS

FIG. 1 is a schematic cross-sectional view schematically illustrating an example of the configuration of a gas sensor 100.

FIG. 2 is a top view of a pump outer electrode 23p and a voltage outer electrode 23s.

FIG. 3 is a block diagram illustrating an electrical connection relationship between a control device 95 and the cells of a sensor element 101.

FIG. 4 shows graphs illustrating the change in response time of voltage Vref before and after a continuous test in atmosphere.

FIG. 5 shows graphs illustrating the manner of temporal change in voltage Vref in Example 1 and Comparative Example 1 after a continuous test in atmosphere.

FIG. 6 is an explanatory chart illustrating an example of temporal change in voltage Vp3.

FIG. 7 is an explanatory chart illustrating an example of temporal change in voltage Vref.

FIG. 8 is a schematic cross-sectional view of a gas sensor 200 according to a modification.

FIG. 9 is a schematic cross-sectional view schematically illustrating an example of a gas sensor 900 as a conventional example.

FIG. 10 is a partial cross-sectional view illustrating a diffusion layer 26 that covers the pump outer electrode 23p.

### DETAILED DESCRIPTION OF THE INVENTION

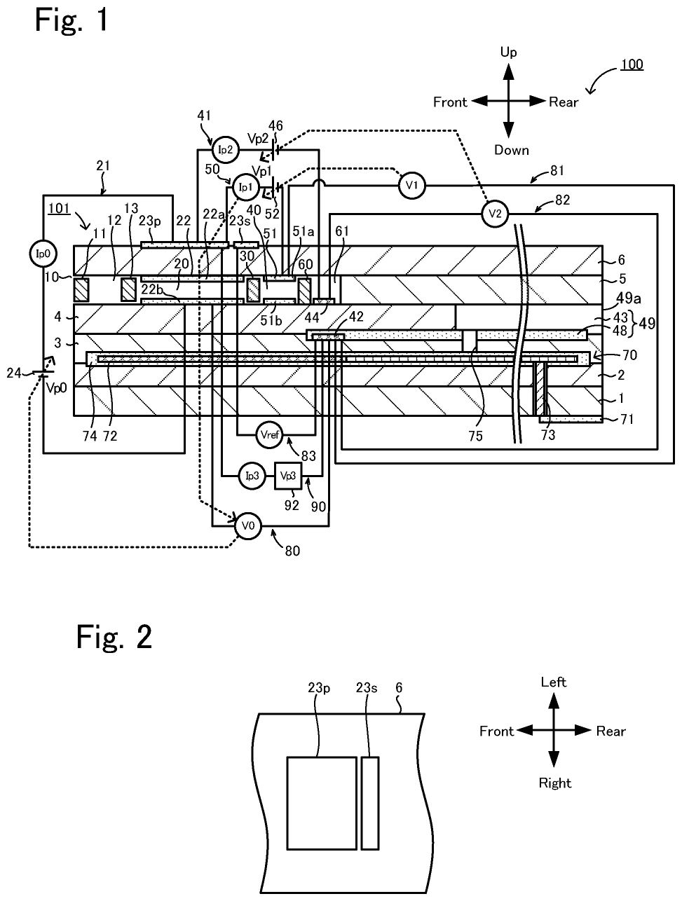

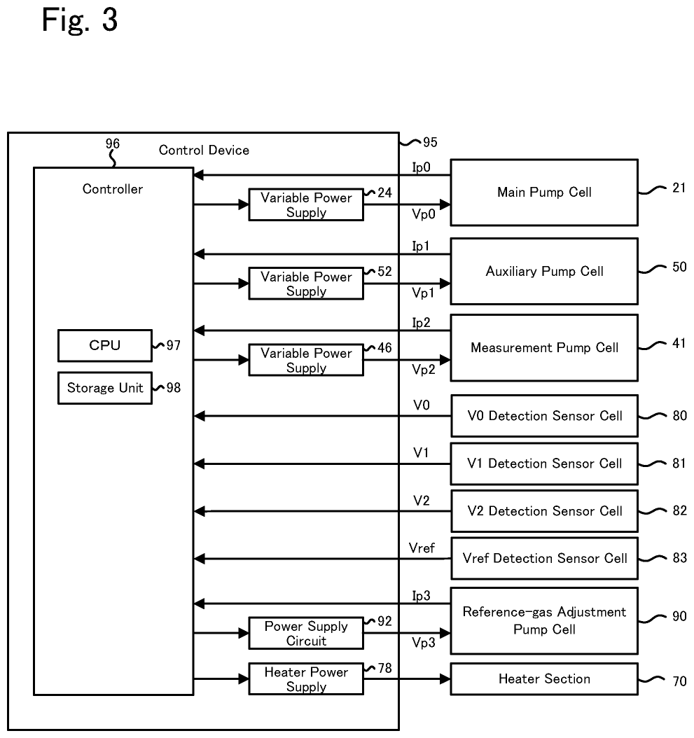

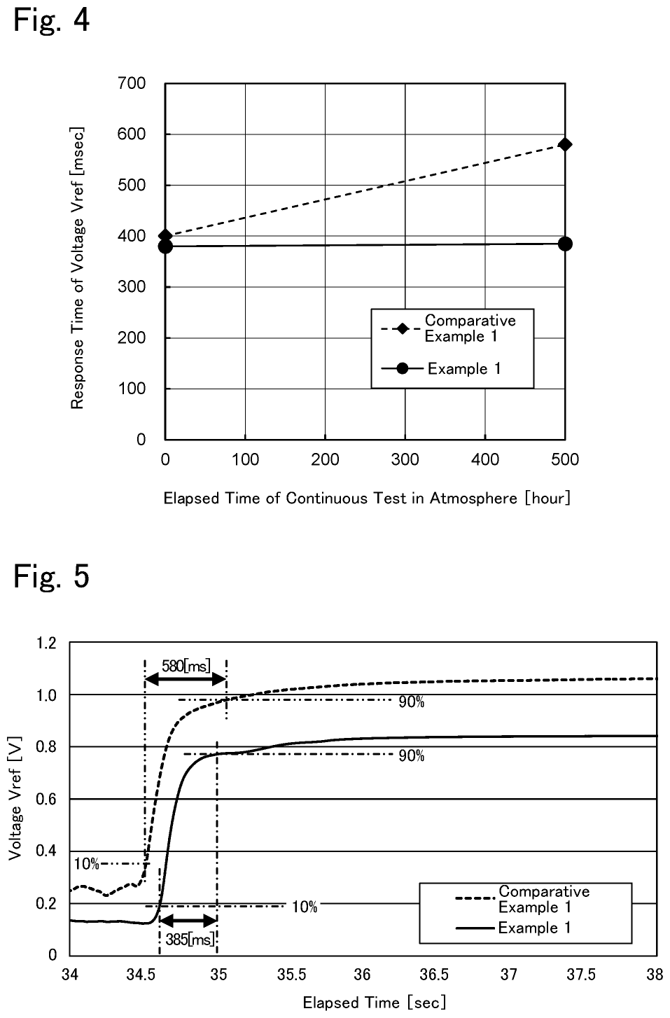

Next, an embodiment of the present invention will be described using the drawings. FIG. 1 is a schematic cross-sectional view schematically illustrating an example of the configuration of a gas sensor 100 in an embodiment of the present invention. FIG. 2 is a top view of a pump outer electrode 23p and a voltage outer electrode 23s of a sensor element 101. FIG. 3 is a block diagram illustrating an electrical connection relationship between a control device and the cells of the sensor element 101. The gas sensor 100 includes: the sensor element 101 having an elongate rectangular parallelepiped shape; and the control device 95 that controls the entire gas sensor 100. The gas sensor 100 also includes: an element sealing body (not illustrated) that seals and fixes the sensor element 101; and a bottomed cylindrical protective cover (not illustrated) that protects the front end of the sensor element 101. The sensor element 101 includes cells 21, 41, 50, 80 to 83, 90 and a heater section 70.

The gas sensor 100 is mounted on a pipe such as the exhaust gas pipe of an internal combustion engine, for example. The gas sensor 100 detects the concentration of a specific gas such as NOx and ammonia in a measurement-object gas which is an exhaust gas of an internal combustion engine. In this embodiment, the gas sensor 100 measures the NOx concentration as the specific gas concentration. The longitudinal direction (i.e., the left-right direction in FIG. 1) of the sensor element 101 is defined as the front-rear direction, and the thickness direction (i.e., the up-down direction in FIG. 1) of the sensor element 101 is defined as the up-down direction. Furthermore, the width direction (i.e., the direction perpendicular to the front-rear direction and the up-down direction) of the sensor element 101 is defined as the left-right direction.

As illustrated in FIG. 1, the sensor element 101 has a layered body obtained by stacking six layers, namely, a first substrate layer 1, a second substrate layer 2, a third substrate layer 3, a first solid electrolyte layer 4, a spacer layer 5, and a second solid electrolyte layer 6 that are formed of oxygen-ion-conductive solid electrolyte layers composed of, for example, zirconia (ZrO₂), in that order from below in the drawing. The solid electrolyte used for forming each of these six layers is dense and hermetic. For example, the sensor element 101 is manufactured by performing predetermining processing and printing of a circuit pattern on ceramic green sheets corresponding to the individual layers, subsequently stacking the sheets, and then combining the sheets by calcination.

On the leading end side (front end side) of the sensor element 101 and between the lower surface of the second solid electrolyte layer 6 and the upper surface of the first solid electrolyte layer 4, a gas inlet 10, a first diffusion control section 11, a buffer space 12, a second diffusion control section 13, a first internal cavity 20, a third diffusion control section 30, a second internal cavity 40, a fourth diffusion control section 60, and a third internal cavity 61 are adjacently formed in that order to communicate with each other.

The gas inlet 10, the buffer space 12, the first internal cavity 20, the second internal cavity 40, the third internal cavity 61 form a space inside the sensor element 101, the space being provided by hollowing out the spacer layer 5 and partitioning the upper part of the space by the lower surface of the second solid electrolyte layer 6, the lower part by the upper surface of the first solid electrolyte layer 4, and the lateral part by the lateral surface of the spacer layer 5.

The first diffusion control section 11, the second diffusion control section 13, and the third diffusion control section 30 are each provided as two horizontally long slits (with an opening having a longitudinal direction in the direction perpendicular to the drawing). In addition, the fourth diffusion control section 60 is provided as one horizontally long slit (with an opening having a longitudinal direction in the direction perpendicular to the drawing) formed as a gap from the lower surface of the second solid electrolyte layer 6. Note that the portion from the gas inlet 10 to the third internal cavity 61 is also referred to as the measurement-object gas flow portion.

The sensor element 101 includes a reference-gas introduction portion 49 that causes a reference gas for measuring the NOx concentration to flow through a reference electrode 42 from the outside of the sensor element 101. The reference-gas introduction portion 49 has a reference-gas introduction space 43, and a reference-gas introduction layer 48. The reference-gas introduction space 43 is a space provided inwardly from the rear end surface of the sensor element 101. The reference-gas introduction space 43 is provided between the upper surface of the third substrate layer 3 and the lower surface of the spacer layer 5, and at the position where the lateral part is partitioned by the lateral surface of the first solid electrolyte layer 4. The reference-gas introduction space 43 has an opening in the rear end surface of the sensor element 101, and a reference gas is introduced into the reference-gas introduction space 43 through the opening. The reference-gas introduction portion 49 guides the reference gas introduced from the outside of the sensor element 101 to the reference electrode 42, while adding a predetermined diffusion resistance to the reference gas. In this embodiment, the reference gas is an atmospheric gas.

The reference-gas introduction layer 48 is provided between the upper surface of the third substrate layer 3 and the lower surface of the first solid electrolyte layer 4. The reference-gas introduction layer 48 is a porous body composed of ceramics such as alumina. Part of the upper surface of the reference-gas introduction layer 48 is exposed to the reference-gas introduction space 43. The reference-gas introduction layer 48 is formed to cover the reference electrode 42. The reference-gas introduction layer 48 causes the reference gas to flow from the reference-gas introduction space 43 to the reference electrode 42. The reference-gas introduction portion 49 does not need to include the reference-gas introduction space 43. In that case, the reference-gas introduction layer 48 should be exposed to the rear end surface of the sensor element 101.

The reference electrode 42 is interposed between the upper surface of the third substrate layer 3 and the first solid electrolyte layer 4, and as described above, in the periphery of the reference electrode 42, the reference-gas introduction layer 48 connected to the reference-gas introduction space 43 is provided. Furthermore, as will be described later, with the reference electrode 42, it is possible to measure the oxygen concentration (oxygen partial pressure) in the first internal cavity 20, the second internal cavity 40, and the third internal cavity 61. The reference electrode 42 is formed as a porous cermet electrode (e.g., a cermet electrode composed of Pt and ZrO₂).

In the measurement-object gas flow portion, the gas inlet 10 is a portion which is opened to the exterior space, and is designed to take the measurement-object gas into the sensor element 101 from the exterior space through the gas inlet 10. The first diffusion control section 11 adds a predetermined diffusion resistance to the measurement-object gas taken through the gas inlet 10. The buffer space 12 is provided to guide the measurement-object gas introduced from the first diffusion control section 11 to the second diffusion control section 13. The second diffusion control section 13 adds a predetermined diffusion resistance to the measurement-object gas introduced from the buffer space 12 into the first internal cavity 20. When the measurement-object gas is introduced from the outside of the sensor element 101 into the first internal cavity 20, the measurement-object gas suddenly taken into the sensor element 101 through the gas inlet 10 by a pressure variation (pulsation of the exhaust gas pressure when the measurement-object gas is exhaust gas of an automobile) of the measurement-object gas in the exterior space is not directly introduced into the first internal cavity 20, but is introduced into the first internal cavity 20 after the pressure variation in the measurement-object gas is cancelled through the first diffusion control section 11, the buffer space 12, and the second diffusion control section 13. Consequently, the pressure variation in the measurement-object gas introduced into the first internal cavity 20 is almost negligible. The first internal cavity 20 is provided as a space to adjust the oxygen partial pressure in the measurement-object gas introduced through the second diffusion control section 13. The oxygen partial pressure is adjusted by the main pump cell 21 operating. The main pump cell 21 is an electrochemical pump cell including: an inner pump electrode 22 having a ceiling electrode portion 22a provided on substantially the entire lower surface of the second solid electrolyte layer 6 facing the first internal cavity 20; a pump outer electrode 23p provided to be exposed to the exterior space in an area corresponding to the ceiling electrode portion 22a on the upper surface of the second solid electrolyte layer 6; and the second solid electrolyte layer 6 interposed by these electrodes.

The inner pump electrode 22 is formed across the upper and lower solid electrolyte layers (the second solid electrolyte layer 6 and the first solid electrolyte layer 4) defining the first internal cavity 20, and the spacer layer that provides a sidewall. Specifically, the ceiling electrode portion 22a is formed on the lower surface of the second solid electrolyte layer 6 providing the ceiling surface of the first internal cavity 20, a bottom electrode portion 22b is formed on the upper surface of the first solid electrolyte layer 4 providing the bottom surface, and a lateral electrode portion (not illustrated) is formed on the lateral wall surface (inner surface) of the spacer layer forming both sidewalls of the first internal cavity 20 so as to connect the ceiling electrode portion 22a and the bottom electrode portion 22b, so that these electrodes are disposed in a structure of a tunnel form at the arrangement position of the lateral electrode portion.

The inner pump electrode 22 is formed as a porous cermet electrode (e.g., a cermet electrode composed of Pt containing 1% of Au and ZrO₂). Note that the inner pump electrode 22 to be in contact with the measurement-object gas is formed using a material having a decreased reducing ability for NOx component in the measurement-object gas.

In the main pump cell 21, oxygen in the first internal cavity 20 can be pumped out to the exterior space or oxygen in the exterior space can be pumped into the first internal cavity 20 by applying a desired voltage Vp0 across the inner side pump electrode 22 and the pump outer electrode 23p to cause a pump current Ip0 to flow in a positive direction or a negative direction between the inner side pump electrode 22 and the pump outer electrode 23p.

Furthermore, in order to detect the oxygen concentration (oxygen partial pressure) in an atmosphere in the first internal cavity 20, an electrochemical sensor cell, that is, a V0 detection sensor cell 80 (also referred to as an oxygen partial pressure detection sensor cell for main pump control) is formed by the inner pump electrode 22, the second solid electrolyte layer 6, the spacer layer 5, the first solid electrolyte layer 4, the third substrate layer 3, and the reference electrode 42.

The oxygen concentration (oxygen partial pressure) in the first internal cavity 20 can be found by measuring the voltage V0 in the V0 detection sensor cell 80.

Furthermore, the pump current Ip0 is controlled by feedback-controlling the voltage Vp0 of a variable power supply 24 so that the voltage V0 reaches a target value. Thus, the oxygen concentration in the first internal cavity 20 can be maintained at a predetermined constant value. The voltage V0 is a voltage across the inner pump electrode 22 and the reference electrode 42.

The third diffusion control section 30 adds a predetermined diffusion resistance to the measurement-object gas whose oxygen concentration (oxygen partial pressure) is controlled by the operation of the main pump cell 21 in the first internal cavity 20, and introduces the measurement-object gas to the second internal cavity 40.

After the oxygen concentration (oxygen partial pressure) is adjusted in advance in the first internal cavity 20, the second internal cavity 40 is provided as a space for further adjusting the oxygen partial pressure, by the auxiliary pump cell 50, of the measurement-object gas introduced through the third diffusion control section 30. Therefore, the oxygen concentration in the second internal cavity 40 can be maintained at a constant level with high accuracy, thus highly accurate measurement of NOx concentration is made possible in the gas sensor 100.

The auxiliary pump cell 50 is an auxiliary electrochemical pump cell including: an auxiliary pump electrode 51 having a ceiling electrode portion 51a provided on substantially the entire lower surface of the second solid electrolyte layer 6 facing the second internal cavity 40; the pump outer electrode 23p; and the second solid electrolyte layer 6.

The auxiliary pump electrode 51 is disposed in the second internal cavity 40 in a structure of a tunnel form as in the inner pump electrode 22 provided in the aforementioned first internal cavity 20. Specifically, the ceiling electrode portion 51a is formed for the second solid electrolyte layer 6 that provides the ceiling surface of the second internal cavity 40, the bottom electrode portion 51b is formed for the first solid electrolyte layer 4 that provides the bottom surface of the second internal cavity 40, and a lateral electrode portion (not illustrated) that connects the ceiling electrode portion 51a and the bottom electrode portion 51b is formed in each of both wall surfaces of the spacer layer 5, which provide the lateral wall of the second internal cavity 40, thereby implementing a structure of a tunnel form. Note that as in the inner pump electrode 22, the auxiliary pump electrode 51 is also formed using a material having a decreased reducing ability for NOx component in the measurement-object gas.

In the auxiliary pump cell 50, oxygen in an atmosphere in the second internal cavity 40 can be pumped out to the exterior space or oxygen can be pumped from the exterior space into the second internal cavity 40 by applying a desired voltage Vp1 across the auxiliary pump electrode 51 and the pump outer electrode 23p.

Furthermore, in order to control the oxygen partial pressure in an atmosphere in the second internal cavity 40, an electrochemical sensor cell, that is, a V1 detection sensor cell 81 (also referred to as an auxiliary-pump-control oxygen-partial-pressure detection sensor cell) is formed by the auxiliary pump electrode 51, the reference electrode 42, the second solid electrolyte layer 6, the spacer layer 5, the first solid electrolyte layer 4, and the third substrate layer 3.

Note that the auxiliary pump cell 50 performs pumping using a variable power supply 52 whose voltage is controlled based on the voltage V1 detected by the V1 detection sensor cell 81. Thus, the oxygen partial pressure in an atmosphere in the second internal cavity 40 is controlled at a low partial pressure which has substantially no effect on measurement of NOx. The voltage V1 is a voltage across the auxiliary pump electrode 51 and the reference electrode 42.

Along with this, the pump current Ip1 is used to control the electromotive force of the V0 detection sensor cell 80. Specifically, the pump current Ip1 is input to the V0 detection sensor cell 80 as a control signal, and the aforementioned target value of the voltage V0 is controlled so that the slope of the oxygen partial pressure in the measurement-object gas introduced from the third diffusion control section 30 into the second internal cavity 40 is controlled at a constant level all the time. When the gas sensor 100 is used as an NOx sensor, the oxygen concentration in the second internal cavity 40 is maintained at a constant value around approximately 0.001 ppm by the operation of the main pump cell 21 and the auxiliary pump cell 50.

The fourth diffusion control section 60 adds a predetermined diffusion resistance to the measurement-object gas whose oxygen concentration (oxygen partial pressure) is controlled by the operation of the auxiliary pump cell 50 in the second internal cavity 40, and introduces the measurement-object gas to the third internal cavity 61. The fourth diffusion control section 60 has a function of regulating the amount of NOx which flows into the third internal cavity 61.

After the oxygen concentration (oxygen partial pressure) is adjusted in advance in the second internal cavity 40, the third internal cavity 61 is provided as a space to perform a process related to measurement of the nitrogen oxide (NOx) concentration in the measurement-object gas on the measurement-object gas introduced through the fourth diffusion control section 60. The NOx concentration is mainly measured by the operation of the measurement pump cell 41 in the third internal cavity 61.

The measurement pump cell 41 measures the NOx concentration in the measurement-object gas in the third internal cavity 61. The measurement pump cell 41 is an electrochemical pump cell including: a measurement electrode 44 provided on the upper surface of the first solid electrolyte layer 4 facing the third internal cavity 61; the pump outer electrode 23p; the second solid electrolyte layer 6; the spacer layer 5; and the first solid electrolyte layer 4. The measurement electrode 44 is a porous cermet electrode including a material which has a higher reducing ability for NOx component in the measurement-object gas than the reducing ability of the inner pump electrode 22. The measurement electrode 44 also functions as an NOx reduction catalyst to reduce the NOx present in an atmosphere in the third internal cavity 61.

In the measurement pump cell 41, the oxygen generated by decomposition of nitrogen oxide in an atmosphere in the periphery of the measurement electrode 44 is pumped out, and the amount of generated oxygen can be detected as the pump current Ip2.

In order to detect the oxygen partial pressure in the periphery of the measurement electrode 44, an electrochemical sensor cell, that is, a V2 detection sensor cell 82 (also referred to as a measurement-pump-control oxygen-partial-pressure detection sensor cell) is formed by the first solid electrolyte layer 4, the third substrate layer 3, the measurement electrode 44, and the reference electrode 42. A variable power supply 46 is controlled based on the voltage V2 detected by the V2 detection sensor cell 82. The voltage V2 is a voltage across the measurement electrode 44 and the reference electrode 42.

The measurement-object gas introduced into the second internal cavity 40 reaches the measurement electrode 44 in the third internal cavity 61 through the fourth diffusion control section 60 in a situation where the oxygen partial pressure is controlled. The nitrogen oxide in the measurement-object gas in the periphery of the measurement electrode 44 is reduced (2NO→N₂+O₂) to produce oxygen. The produced oxygen is then pumped by the measurement pump cell 41, and in this process, voltage Vp2 of the variable power supply 46 is controlled so that the voltage V2 detected by the V2 detection sensor cell 82 is constant (target value). The amount of oxygen produced in the periphery of the measurement electrode 44 is in proportion to the concentration of nitrogen oxide in the measurement-object gas, thus the nitrogen oxide concentration in the measurement-object gas is calculated using the pump current Ip2 in the measurement pump cell 41.

An electrochemical Vref detection sensor cell 83 is formed by the second solid electrolyte layer 6, the spacer layer 5, the first solid electrolyte layer 4, the third substrate layer 3, the voltage outer electrode 23s, and the reference electrode 42, and the oxygen partial pressure in the measurement-object gas outside the sensor is detectable with the voltage Vref obtained by the Vref detection sensor cell 83. The voltage Vref is a voltage across the voltage outer electrode 23s and the reference electrode 42.

Furthermore, an electrochemical reference-gas adjustment pump cell 90 is formed by the second solid electrolyte layer 6, the spacer layer 5, the first solid electrolyte layer 4, the third substrate layer 3, the pump outer electrode 23p, and the reference electrode 42. The reference-gas adjustment pump cell 90 pumps oxygen by flowing the pump current Ip3 using a control voltage (voltage Vp3) applied by a power supply circuit 92 connected between the pump outer electrode 23p and the reference electrode 42. Thus, the reference-gas adjustment pump cell 90 pumps oxygen from the space around the pump outer electrode 23p into the periphery of the reference electrode 42.

In the gas sensor 100 having such a configuration, the measurement-object gas having an oxygen partial pressure always maintained at a constant low value (a value having substantially no effect on measurement of NOx) is provided to the measurement pump cell 41 by operating the main pump cell 21 and the auxiliary pump cell 50. Therefore, the NOx concentration in the measurement-object gas can be found based on the pump current Ip2 which flows by pumping-out of oxygen by the measurement pump cell 41, the oxygen being produced by reduction of NOx in amount approximately proportional to the concentration of NOx in the measurement-object gas.

Furthermore, in order to enhance oxygen ion conductivity of the solid electrolyte, the sensor element 101 includes a heater section 70 having a role of temperature adjustment for heating the sensor element 101 and maintaining its temperature. The heater section 70 includes a heater connector electrode 71, a heater 72, a through-hole 73, a heater insulation layer 74, and a pressure diffusion hole 75.

The heater connector electrode 71 is formed to be in contact with the lower surface of the first substrate layer 1. Connecting the heater connector electrode 71 and an external power supply makes it possible to supply power to the heater section 70 from the outside.

The heater 72 is an electrical resistor which is formed to be interposed vertically between the second substrate layer 2 and the third substrate layer 3. The heater 72 is coupled to the heater connector electrode 71 via the through-hole 73, generates heat by being supplied with power from the outside through the heater connector electrode 71, and heats and maintains the temperature of the solid electrolyte forming the sensor element 101.

The heater 72 is buried over the entire region from the first internal cavity 20 to the third internal cavity 61, and the entire sensor element 101 can be adjusted to a temperature at which the solid electrolyte is activated.

The heater insulation layer 74 is composed of an insulator such as alumina on the upper and lower surfaces of the heater 72. The heater insulation layer 74 is formed for the purpose of obtaining an electrical insulating property between the second substrate layer 2 and the heater 72 as well as an electrical insulating property between the third substrate layer 3 and the heater 72.

The pressure diffusion hole 75 is a section provided to penetrate the third substrate layer 3 and the reference-gas introduction layer 48 so as to communicate with the reference-gas introduction space 43, and is formed for the purpose of reducing an internal pressure rise accompanied by a temperature increase in the heater insulation layer 74.

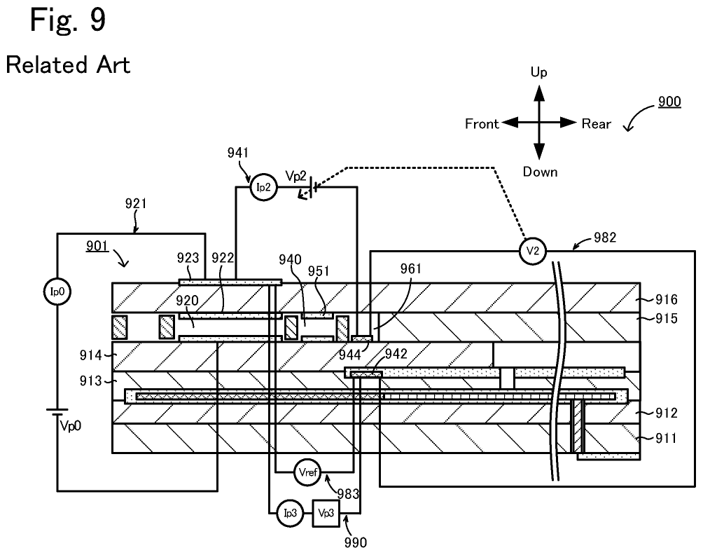

Here, the pump outer electrode 23p and the voltage outer electrode 23s will be described in detail. The pump outer electrode 23p and the voltage outer electrode 23s corresponds to an aspect in which the outer pump electrode 923 in FIG. 9 is divided into two electrodes. Specifically, the outer pump electrode 923 in FIG. 9 serves as the electrode of the main pump cell 921 through which the pump current Ip0 is passed, the electrode of the measurement pump cell 941 through which the pump current Ip2 is passed, the electrode of the reference-gas adjustment pump cell 990 through which the pump current Ip3 is passed, and the electrode of the Vref detection sensor cell 983 that detects the voltage Vref. In contrast, in this embodiment, the pump outer electrode 23p of the main pump cell 21, the auxiliary pump cell 50, the measurement pump cell 41, and the reference-gas adjustment pump cell 90, and the voltage outer electrode 23s of the Vref detection sensor cell 83 are both disposed outside the sensor element 101 as independent electrodes.

In this embodiment, as illustrated in FIG. 2, the pump outer electrode 23p and the voltage outer electrode 23s each have an approximately quadrangle shape in a top view. The voltage outer electrode 23s is located rearward of the pump outer electrode 23p. The voltage outer electrode 23s is shorter in length in the front-rear direction and smaller in area than the pump outer electrode 23p. Note that the area of an electrode is the one as seen in the direction perpendicular to the surface where the electrode is disposed. For example, the areas of the pump outer electrode 23p and the voltage outer electrode 23s are each an area in a top view.

The pump outer electrode 23p and the voltage outer electrode 23s each contain a noble metal (e.g., at least one of Pt, Rh, Pd, Ru and Ir) having catalytic activity. The pump outer electrode 23p and the voltage outer electrode 23s are preferably composed of a cermet containing a noble metal and an oxide (in this case, ZrO₂) having oxygen ion conductivity. The pump outer electrode 23p and the voltage outer electrode 23s are preferably a porous body. The noble metal contained in the pump outer electrode 23p and the noble metal contained in the voltage outer electrode 23s may be the same in each of type and content ratio, or may be different in at least one of type and content ratio. In this embodiment, the pump outer electrode 23p and the voltage outer electrode 23s are each a porous cermet electrode composed of Pt and ZrO₂.

As illustrated in FIG. 3, the control device 95 includes the aforementioned variable power supplies 24, 46, 52, a heater power supply 78, the aforementioned power supply circuit 92, and a controller 96. The controller 96 is a microprocessor including a CPU 97, a RAM which is not illustrated, and a storage unit 98. The storage unit 98 is, for example, a non-volatile memory such as a ROM, which is a device that stores various data. The controller 96 receives inputs of the voltages V0 to V2 and the voltage Vref of the sensor cells 80 to 83. The controller 96 receives inputs of the pump currents Ip0 to Ip3 which flow the respective pump cells 21, 50, 41, 90. The controller 96 controls the voltages Vp0 to Vp3 output by the variable power supplies 24, 46, 52 and the power supply circuit 92 by outputting a control signal to the variable power supplies 24, 46, 52 and the power supply circuit 92, thereby controlling the pump cells 21, 41, 50, 90. The controller 96 controls the electric power to be supplied to the heater 72 by the heater power supply 78 by outputting a control signal to the heater power supply 78, thereby adjusting the temperature of the sensor element 101. The storage unit 98 stores the target value V0*, V1*, V2*, Ip1* mentioned below.

The controller 96 feedback-controls the voltage Vp0 of the variable power supply 24 so that the voltage V0 reaches a target value V0* (in other words, so that the oxygen concentration in the first internal cavity 20 reaches a target concentration).

The controller 96 feedback-controls the voltage Vp1 of the variable power supply 52 so that the voltage V1 reaches a constant value (referred to as a target value V1*) (in other words, so that the oxygen concentration in the second internal cavity 40 reaches a predetermined low oxygen concentration which has substantially no effect on measurement of NOx). Along with this, the controller 96 sets (feedback-controls) the target value V0* of the voltage V0 based on the pump current Ip1 so that the pump current Ip1 caused to flow by the voltage Vp1 reaches a constant value (referred to as a target value Ip1*). Consequently, the slope of the oxygen partial pressure in the measurement-object gas introduced from the third diffusion control section 30 into the second internal cavity 40 becomes constant all the time. In addition, the oxygen partial pressure in an atmosphere in the second internal cavity 40 is controlled at a low partial pressure which has substantially no effect on measurement of NOx. The target value V0* is set to a value that causes the oxygen concentration in the first internal cavity 20 to be higher than 0% and reach a low oxygen concentration.

The controller 96 feedback-controls the voltage Vp2 of the variable power supply 46 so that the voltage V2 reaches a constant value (referred to as a target value V2*) (in other words, the oxygen concentration in the third internal cavity 61 reaches a predetermined low concentration). Thus, oxygen is pumped out from the third internal cavity 61 so that the oxygen produced by reducing the specific gas (in this case, NOx) in the measurement-object gas in the third internal cavity 61 becomes substantially zero. The controller 96 then obtains the pump current Ip2 as a detection value corresponding to the oxygen produced from NOx in the third internal cavity 61, and calculates the NOx concentration in the measurement-object gas based on the pump current Ip2. The target value V2* is a predetermined value such that the pump current Ip2 caused to flow by the feedback-controlled voltage Vp2 becomes a limiting current. The storage unit 98 stores a relational expression (e.g., the expression of a linear function) and a map as a correspondence relationship between the pump current Ip2 and the NOx concentration. Such a relational expression and a map can be determined by an experiment in advance. The controller 96 then detects the NOx concentration in the measurement-object gas based on the obtained pump current Ip2 and the aforementioned correspondence relationship stored in the storage unit 98. In this manner, oxygen from the specific gas in the measurement-object gas introduced into the sensor element 101 is pumped out, and the specific gas concentration is detected based on the amount of oxygen pumped out (based on the pump current Ip2 in this embodiment). This method is referred to as a limiting current method.

The controller 96 causes the pump current Ip3 to flow by controlling the power supply circuit 92 so that the voltage Vp3 is applied to the reference-gas adjustment pump cell 90. The flowing of the pump current Ip3 causes the reference-gas adjustment pump cell 90 to pump in oxygen from the periphery of the pump outer electrode 23p to the periphery of the reference electrode 42.

The function of the reference-gas adjustment pump cell 90 will be described below. The measurement-object gas which has flowed into the aforementioned protective cover (not illustrated) is introduced to a measurement-object gas flow portion, such as the gas inlet 10, of the sensor element 101. In contrast, a reference gas (atmosphere) is introduced to the reference-gas introduction portion 49 of the sensor element 101. The gas inlet 10 side of the sensor element 101 and the entry side of the reference-gas introduction portion 49, in short, the front end side and the rear end side of the sensor element 101 are partitioned and sealed by the aforementioned element sealing body (not illustrated) to prevent flow of gas between the sides. However, when the pressure on the side of measurement-object gas is high, the measurement-object gas may slightly enter the reference-gas side, and the oxygen concentration of the reference gas in the periphery of the rear end side of the sensor element 101 may decrease. At this point, if the oxygen concentration in the periphery of the reference electrode 42 also decreases, the reference potential which is the electrical potential of the reference electrode 42 also changes. The voltages V0 to V2, Vref of the sensor cells 80 to 83 mentioned above are each a voltage relative to the electrical potential of the reference electrode 42, thus when the reference potential changes, the accuracy of detection of the NOx concentration in the measurement-object gas may decrease. The reference-gas adjustment pump cell 90 serves to prevent such decrease in the detection accuracy. The control device 95 controls the power supply circuit 92, and applies, as the voltage Vp3, a pulse voltage repeatedly turned ON and OFF with a predetermined cycle (e.g., 10 msec) across the reference electrode 42 and the pump outer electrode 23p of the reference-gas adjustment pump cell 90. The flowing of the pump current Ip3 through the reference-gas adjustment pump cell 90 caused by the voltage Vp3 allows oxygen to be pumped in from the periphery of the pump outer electrode 23p to the periphery of the reference electrode 42. Consequently, as described above, when the measurement-object gas causes the oxygen concentration to decrease in the periphery of the reference electrode 42, the decreased oxygen can be supplemented, and reduction in the accuracy of detection of the NOx concentration can be prevented.

Note that in addition to the variable power supplies 24, 46, 52, the heater power supply 78 and the power supply circuit 92 which are illustrated in FIG. 3, the control device 95 is actually connected to the electrodes inside the sensor element 101 through unillustrated lead wires formed in the sensor element 101, and unillustrated connector electrodes (only the heater connector electrode 71 is illustrated in FIG. 1) formed on the rear end side of the sensor element 101.

The process performed by the controller 96 at the time of detection of the NOx concentration in the measurement-object gas by the gas sensor 100 will be described. First, the CPU 97 of the controller 96 starts to drive the sensor element 101. Specifically, the CPU 97 transmits a control signal to the heater power supply 78 to heat the sensor element 101 by the heater 72. The CPU 97 then heats the sensor element 101 to a predetermined driving temperature (e.g., 800° C.). Next, the CPU 97 starts to control the aforementioned pump cells 21, 41, 50, 90, and obtain the voltages V0 to V2, Vref from the aforementioned sensor cells 80 to 83. When the measurement-object gas is introduced through the gas inlet 10 in this state, the measurement-object gas passes through the first diffusion control section 11, the buffer space 12 and the second diffusion control section 13, and reaches the first internal cavity 20. Next, the oxygen concentration of the measurement-object gas is adjusted by the main pump cell 21 and the auxiliary pump cell 50 in the first internal cavity and the second internal cavity 40, and the measurement-object gas after the adjustment reaches the third internal cavity 61. The CPU 97 then detects the NOx concentration in the measurement-object gas based on the obtained pump current Ip2 and the correspondence relationship stored in the storage unit 98.

In the sensor element 101 of the gas sensor 100, as described above, the pump outer electrode 23p constituting part of each of the pump cells 21, 41, 50, 90, and the voltage outer electrode 23s constituting part of the Vref detection sensor cell 83 are each disposed outside the sensor element 101. In other words, in the sensor element 101, the pump outer electrode 23p and the voltage outer electrode 23s are separately provided outside the sensor element 101. Thus, unlike when one electrode serves as the pump outer electrode 23p and the voltage outer electrode 23s (e.g., in the sensor element 901 illustrated in FIG. 9, the outer pump electrode 923 serves as the electrode of the main pump cell 921, the electrode of the measurement pump cell 941, the electrode of the reference-gas adjustment pump cell 990, and the electrode of the Vref detection sensor cell 983), the pump currents Ip0 to Ip3 of the main pump cell 21, the measurement pump cell 41, the auxiliary pump cell 50, and the reference-gas adjustment pump cell 90 do not flow through the voltage outer electrode 23s. Therefore, the voltage Vref of the Vref detection sensor cell 83 does not include a voltage drop of the voltage outer electrode 23s due to the pump currents Ip0 to Ip3. Consequently, the voltage Vref of the Vref detection sensor cell 83 has a value which corresponds with higher accuracy to the oxygen concentration in the measurement-object gas outside the sensor element 101, thus the accuracy of detection of the oxygen concentration in the measurement-object gas using the Vref detection sensor cell 83 is improved.

Note that when one outer pump electrode 923 is provided and the pump outer electrode 23p and the voltage outer electrode 23s are not independent as in the sensor element 901 in a conventional example, in addition to the electromotive force based on the oxygen concentration difference between the periphery of the outer pump electrode 923 and the periphery of the reference electrode 942, the voltage Vref of the Vref detection sensor cell 983 includes the value (voltage drop) obtained by multiplying the total value of the pump currents flowing through the outer pump electrode 923 by the resistance of the outer pump electrode 923. Regarding the magnitude of a voltage drop in the outer pump electrode 923, due to the effect of a manufacturing variation (e.g., a variation in state, such as thickness, degree of porosity, surface area) of the outer pump electrode 923, when multiple sensor elements 901 are manufactured, individual difference may occur for each sensor element 901. Thus, in the sensor element 901, the accuracy of detection of the oxygen concentration outside the sensor element 901 using the voltage Vref may have a variation for each sensor element 901. In contrast, in the sensor element 101 in this embodiment, a voltage drop does not occur in the voltage outer electrode 23s because the pump currents Ip0 to Ip3 are not passed, thus even when a plurality of sensor elements 101 have a manufacturing variation in the voltage outer electrode 23s, the accuracy of detection of the oxygen concentration outside the sensor element 101 using the voltage Vref is unlikely to have a variation.

As described above, the controller 96 controls the main pump cell 21 so that the voltage V0 reaches the target value V0*, in other words, the oxygen concentration in the first internal cavity 20 reaches a predetermined low concentration. In this situation, for example, when the oxygen concentration in the measurement-object gas is switched between a high state in which the oxygen concentration is higher than a predetermined low concentration and a low state, the controller 96 switches the direction of oxygen moved by the main pump cell 21 to the reverse direction. Thus, the direction of the pump current Ip0 which flows through the main pump cell 21 is switched to the reverse direction. For example, when the measurement-object gas is switched from a lean atmosphere to a rich atmosphere, the direction of the pump current Ip0 which flows through the main pump cell 21 is switched from the direction in which oxygen is pumped out from the first internal cavity 20 to the direction in which oxygen is pumped into the first internal cavity 20. The lean atmosphere indicates a state where the air-fuel ratio of the measurement-object gas is higher than a theoretical air-fuel ratio, and the rich atmosphere indicates a state where the air-fuel ratio of the measurement-object gas is lower than a theoretical air-fuel ratio. In a rich atmosphere, the measurement-object gas contains an unburnt fuel, and the right amount of oxygen required for burning the unburnt fuel corresponds to the oxygen concentration in the measurement-object gas in a rich atmosphere. Therefore, the oxygen concentration in the measurement-object gas in a rich atmosphere is expressed as a negative value. Thus, when the measurement-object gas is in a rich atmosphere, in order to change a negative oxygen concentration to a predetermined low concentration (a state where the oxygen concentration is higher than 0%) corresponding to the target value V0*, the controller 96 controls the main pump cell 21 to pump oxygen into the first internal cavity 20. Thus, when one electrode serves as the pump outer electrode 23p as well as the voltage outer electrode 23s, the change in the voltage Vref also becomes slow due to the time required for current change when the direction of the pump current Ip0 flowing through the main pump cell 21 is switched to the reverse direction. In contrast, in this embodiment, the pump outer electrode 23p and the voltage outer electrode 23s are separately provided, thus the voltage Vref is not affected by the time required for change in the pump current Ip0, and therefore, the change in the voltage Vref does not become slow. In other words, when the oxygen concentration in the measurement-object gas is switched between a high state in which the oxygen concentration is higher than a predetermined low concentration and a low state, the responsiveness of the voltage Vref is not likely to reduce.

In addition, when one electrode serves as the pump outer electrode 23p as well as the voltage outer electrode 23s, the electrode deteriorates with use, thus the aforementioned time required for current change when the direction of the pump current Ip0 is switched to the reverse direction may be further increased. This is probably because capacity components of the electrode change due to deterioration of the electrode. Deterioration of an electrode occurs due to oxidation of the noble metal in the electrode caused by flow of a current through the electrode, for example. For example, when the outer pump electrode 923 of the sensor element 901 contains Pt, part of the Pt may be oxidized to produce PtO, PtO₂. Due to deterioration of the outer pump electrode 923, for example, in the gas sensor 900, the responsiveness of the voltage Vref may reduce with use (hereinafter referred to as “deterioration of responsiveness”). In contrast, in this embodiment, the voltage outer electrode 23s is unlikely to deteriorate because the pump currents Ip0 to Ip3 are not passed through the voltage outer electrode 23s. Even if the voltage outer electrode 23s deteriorates, the pump current Ip0 is not passed through the voltage outer electrode 23s, thus the voltage outer electrode 23s is not affected by switching of the direction of the pump current Ip0 to the reverse direction. Consequently, even when the sensor element 101 is used for a long time, the responsiveness of the voltage Vref is unlikely to deteriorate.

The responsiveness of the voltage Vref and the manner of deterioration of the responsiveness have been studied in the following way. First, Example 1 is implemented by producing the sensor element 101 and the gas sensor 100 in this embodiment illustrated in FIGS. 1 to 3. In addition, Comparative Example 1 is implemented by producing a gas sensor which is the same as Example 1 except that the pump outer electrode 23p and the voltage outer electrode 23s are not included but the outer pump electrode 923 of FIG. 9 is included. In Comparative Example 1, the outer pump electrode 923 constitutes part of each of the main pump cell 21, the auxiliary pump cell 50, the measurement pump cell 41, the reference-gas adjustment pump cell 90, and the Vref detection sensor cell 83. The same material is used for the pump outer electrode 23p, and the voltage outer electrode 23s in Example 1, and the outer pump electrode 923 in Comparative Example 1.

For Example 1 and Comparative Example 1, the responsiveness of the voltage Vref was studied. First, the gas sensor in Example 1 was mounted on a pipe. The heater 72 was energized to attain a temperature of 800° C. to heat the sensor element 101. A state is achieved in which the aforementioned pump cells 21, 41, 50 are controlled by the controller 96, and the voltages V0, V1, V2, Vref are obtained from the aforementioned sensor cells 80 to 83. A state is achieved in which the reference-gas adjustment pump cell 90 is not controlled by the controller 96. In this state, as a measurement-object gas, a gas simulating an exhaust gas in a lean state is passed through a pipe, and subsequently, a gas simulating an exhaust gas in a rich state is passed through the pipe, thus switching of the measurement-object gas from a lean state to a rich state was simulated. The voltage Vref then was continuously measured, and the manner of temporal change in the voltage Vref was studied. Similarly, also for Comparative Example 1, the manner of temporal change in the voltage Vref was studied.

Specifically, when the gas to be passed through the pipe is switched from a lean state to a rich state, the voltage Vref rose in each of Example 1 and Comparative Example 1. The value of the voltage Vref immediately before rise thereof is assumed to be 0%, the value of the voltage Vref after being stabilized after the rise is assumed to be 100%, and the response time [msec] of the voltage Vref is defined by the time required for the voltage Vref to change from 10% to 90%. A shorter response time indicates a higher responsiveness of the voltage Vref. The response time in Example 1 was 380 msec, and the response time in Comparative Example 1 was 400 msec. From this result, it was verified that the responsiveness of rising of the voltage Vref is higher in Example 1 in which the pump outer electrode 23p and the voltage outer electrode 23s are both provided than in Comparative Example 1 in which the outer pump electrode 923 is disposed instead of these electrodes. The responsiveness of falling of the voltage Vref at the time of switching the gas to be passed through the pipe from a rich state to a lean state was studied in the same manner, and the responsiveness was higher in Example 1 than in Comparative Example 1.

Next, in a state where the gas sensor 100 in Example 1 was placed in the atmosphere, a continuous test in atmosphere was conducted in the same manner as described above, that is, the sensor element 101 was driven by the controller 96 to operate until 500 hours elapsed. For the gas sensor in Comparative Example 1, a continuous test in atmosphere was also conducted in the same manner. The atmosphere is higher in oxygen concentration than the exhaust gas, and the noble metal in the electrode is likely to be oxidized and deteriorated, thus the continuous test in atmosphere is an accelerated deterioration test for electrode. For Example 1 and Comparative Example 1 after the continuous test in atmosphere was conducted, the response time [msec] of the voltage Vref was measured by the aforementioned method.

FIG. 4 shows graphs illustrating the change in response time of the voltage Vref before and after the continuous test in atmosphere in Example 1 and Comparative Example 1. As illustrated in FIG. 4, in Comparative Example 1, the response time (580 msec) after the continuous test in atmosphere (elapsed time is 500 hours) is longer than the response time (400 msec) before the continuous test in atmosphere (elapsed time is 0 hour), that is, the responsiveness has deteriorated. In contrast, in Example 1, the response time changed from 380 msec to 385 msec only before and after the continuous test in atmosphere, thus change in the response time was little. From this result, it was verified that deterioration of the response time of the voltage Vref with use of the gas sensor is further reduced in Example 1 in which the pump outer electrode 23p and the voltage outer electrode 23s are both provided than in Comparative Example 1 in which the outer pump electrode 923 is disposed instead of these electrodes. FIG. 5 shows graphs illustrating the manner of temporal change in the voltage Vref in Example 1 and Comparative Example 1 after the continuous test in atmosphere. In FIG. 5, the voltages Vref corresponding to 10% and 90% are shown for each of Example 1 and Comparative Example 1, where the value of the voltage Vref immediately before rise thereof is assumed to be 0%, and the value of the voltage Vref after being stabilized after the rise is assumed to be 100%. In addition, in FIG. 5, the value of the aforementioned response time was shown for each of Example 1 and Comparative Example 1, where the response time was measured as the time required for the voltage Vref to change from 10% to 90%.

When the controller 96 detects the oxygen concentration in the measurement-object gas outside the sensor element 101 based on the voltage Vref of the Vref detection sensor cell 83, as a kind of detection of the oxygen concentration, whether the measurement-object gas outside the sensor element 101 is in a rich state or a lean state may be determined based on the voltage Vref. For example, a predetermined threshold to determine whether the voltage Vref is in a rising state or a falling state is pre-stored in the storage unit 98, and the controller 96 may binarize an obtained voltage Vref based on the threshold, and can determine whether the measurement-object gas is in a rich state or a lean state. In this manner, the gas sensor 100 functions not only as an NOx sensor but also as a lambda sensor (air-fuel ratio sensor).

Note that in addition to the aforementioned electromotive force based on the oxygen concentration difference between the periphery of the voltage outer electrode 23s and the periphery of the reference electrode 42, the voltage Vref also includes the thermal electromotive force of the voltage outer electrode 23s. Thus, in order to further improve the accuracy of detection of the oxygen concentration using the Vref detection sensor cell 83, it is preferable to reduce the thermal electromotive force of the voltage outer electrode 23s. For example, a temperature variation in the voltage outer electrode 23s can be reduced by decreasing the area of the voltage outer electrode 23s as much as possible, thus the thermal electromotive force of the voltage outer electrode 23s can be reduced. The voltage outer electrode 23s may have a high resistance value because the pump currents Ip0 to Ip3 do not flow therethrough, thus is more easily reduced in area than the pump outer electrode 23p. In this embodiment, as described above, the area of the voltage outer electrode 23s is made smaller than the area of the pump outer electrode 23p, thus the thermal electromotive force of the voltage outer electrode 23s can be made relatively small.

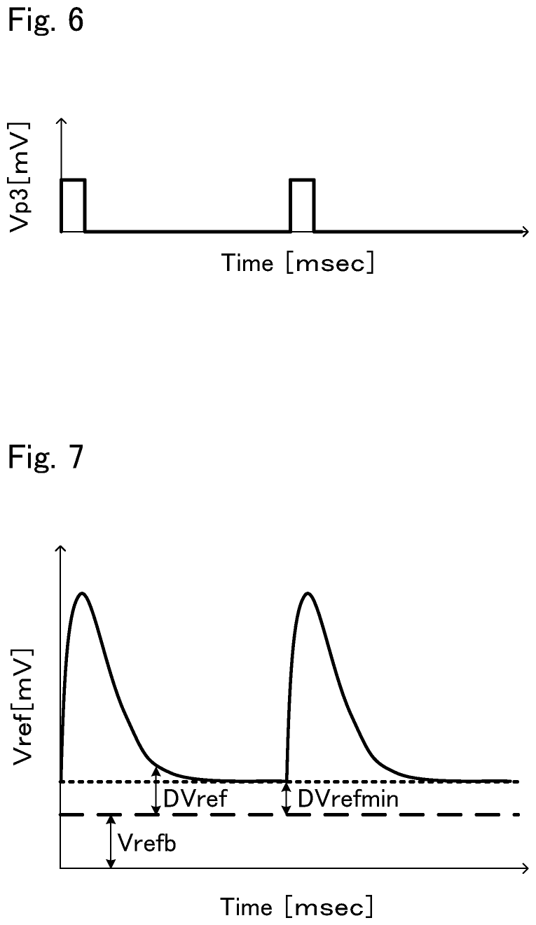

Note that in addition to the aforementioned electromotive force based on the oxygen concentration difference between the periphery of the voltage outer electrode 23s and the periphery of the reference electrode 42, and the thermal electromotive force of the voltage outer electrode 23s, the voltage Vref includes the value (voltage drop) obtained by multiplying the pump current Ip3 of the reference-gas adjustment pump cell 90 by the resistance of the reference electrode 42. In other words, the reference potential that is the electrical potential of the reference electrode 42 changes by the magnitude of a voltage drop thereof occurred according to the pump current Ip3 that flows through the reference electrode 42, and thus the voltage Vref also changes. This will be described. FIG. 6 is an explanatory chart illustrating an example of temporal change in the voltage Vp3. FIG. 7 is an explanatory chart illustrating an example of temporal change in the voltage Vref. When the pulse voltage of FIG. 6 is applied across the reference electrode 42 and the voltage outer electrode 23s as the voltage Vp3, the voltage Vref across the reference electrode 42 and the voltage outer electrode 23s varies like the waveform of FIG. 7. Specifically, when the pulse voltage of the voltage Vp3 is turned ON, the voltage Vref gradually rises accordingly, while when the pulse voltage of the voltage Vp3 is turned OFF, the voltage Vref gradually falls accordingly, and the voltage Vref has a minimum value immediately before the pulse voltage is turned ON subsequently. The reason why the voltage Vref varies in this manner is that the voltage Vref includes a voltage drop caused by the pump current Ip3 that flows through the reference electrode 42. Specifically, rise and fall of the pump current Ip3 is repeated due to the pulse voltage as in the waveform in FIG. 7, thus the magnitude of the voltage drop of the reference electrode 42 also varies according to the pump current Ip3, and the voltage Vref varies like the waveform in FIG. 7. In FIG. 7, the original value (the voltage based on the oxygen concentration difference between the periphery of the reference electrode 42 and the periphery of the pump outer electrode 23p) of the voltage Vref is shown as base voltage Vrefb. Residual voltage DVref that is the difference between the voltage Vref and the base voltage Vrefb includes a voltage drop of the reference electrode 42. The lower the residual voltage DVref, the smaller the change in the electrical potential of the reference electrode 42 due to the pump current Ip3, and the smaller the change in the voltage Vref caused by the change in the electrical potential of the reference electrode 42. Thus, the controller 96 preferably obtains the voltage Vref in a period when the voltage Vp3 is OFF, and more preferably, obtains the voltage Vref at a timing with the residual voltage DVref as low as possible in the OFF-period of the voltage Vp3. In this manner, reduction in the accuracy of measurement of the oxygen concentration in the measurement-object gas outside the sensor element 101, caused by the pump current Ip3 can be prevented and the voltage Vref has a value which corresponds with higher accuracy to the oxygen concentration in the measurement-object gas outside the sensor element 101.

Specifically, the timing with the residual voltage DVref as low as possible may be any timing in the following period. Specifically, first, in one cycle of ON and OFF of the voltage Vp3, the maximum of the value of the voltage Vref is assumed to be 100%, and the minimum is assumed to be 0%. Let the period with a low residual voltage DVref be the period since the voltage Vref falls below 10% after turn-OFF of the voltage Vp3 until the voltage Vref starts to rise due to turn-ON of the voltage Vp3 in the next cycle. The controller 96 preferably obtains the voltage Vref at any timing in this period. More preferably, the controller 96 obtains the voltage Vref at the timing of a minimum DVrefmin (see FIG. 7) of the residual voltage DVref in one cycle of ON and OFF of the voltage Vp3. When the voltage Vref is stable in an OFF-period of the voltage Vp3 (until the voltage Vp3 is turned ON subsequently) as in the waveform of FIG. 7, the controller 96 may obtain the voltage Vref at any timing in the period in which the voltage Vref is stable. In this manner, the controller 96 can obtain the voltage Vref at the timing when the residual voltage DVref attains the minimum DVrefmin. On the other hand, when the voltage Vref is unstable in an OFF-period of the voltage Vp3, the residual voltage DVref attains the minimum DVrefmin at the timing immediately before the subsequent turn-ON in the OFF-period of the voltage Vp3, thus the controller 96 preferably obtains the voltage Vref at this timing. The timing when the controller 96 obtains the voltage Vref can be determined in advance by an experiment based on the ON/OFF cycle of the voltage Vp3, and the waveforms of temporal change in the pump current Ip3 and the voltage Vref caused by the voltage Vp3.

Note that for the sake of explanation, FIG. 7 illustrates the waveform of the voltage Vref when the base voltage Vrefb is constant, specifically, when the oxygen concentration in the measurement-object gas in the periphery of the voltage outer electrode 23s is constant. Actually, the base voltage Vrefb varies, for example, as in FIG. 5 according to the oxygen concentration in the measurement-object gas in the periphery of the voltage outer electrode 23s.

As with the voltage Vref, the voltages V0, V1, V2 are affected by the pump current Ip3. Thus, as with the voltage Vref, the controller 96 obtains the voltages V0, V1, V2 preferably in an OFF-period of the voltage Vp3, more preferably in the aforementioned period with a low residual voltage DVref, and still more preferably at any timing in the period in which the voltage Vref is stable or at the timing immediately before the subsequent turn-ON in an OFF-period of the voltage Vp3. In addition, as with the voltage Vref, the controller 96 obtains the pump currents Ip0 to Ip3 preferably in an OFF-period of the voltage Vp3, more preferably in the aforementioned period with a low residual voltage DVref, and still more preferably at any timing in the period in which the voltage Vref is stable or at the timing immediately before the subsequent turn-ON in an OFF-period of the voltage Vp3. In this embodiment, the controller 96 obtains the voltages V0, V1, V2, Vref, and the pump currents Ip0 to Ip3 at the timing immediately before the subsequent turn-ON in an OFF-period of the voltage Vp3.

The correspondence relationships between the components in this embodiment and the components in the present invention will now be clarified. The first substrate layer 1, the second substrate layer 2, the third substrate layer 3, the first solid electrolyte layer 4, the spacer layer 5 and the second solid electrolyte layer 6 correspond to an element body according to the present invention, the first internal cavity 20 and the second internal cavity 40 correspond to an oxygen concentration adjustment chamber, the pump outer electrode 23p corresponds to a pump outer electrode, the main pump cell 21 and the auxiliary pump cell 50 correspond to an adjustment chamber pump cell, the third internal cavity 61 corresponds to a measurement chamber, the measurement electrode 44 corresponds to a measurement electrode, the measurement pump cell 41 corresponds to a measurement pump cell, the reference-gas introduction portion 49 corresponds to a reference-gas introduction portion, the voltage outer electrode 23s correspond to a voltage outer electrode, the reference electrode 42 corresponds to a reference electrode, and the Vref detection sensor cell 83 corresponds to an outer sensor cell. In addition, the reference-gas adjustment pump cell 90 corresponds to a reference-gas adjustment pump cell, and the controller 96 corresponds to an adjustment chamber pump cell controller, an oxygen concentration detector, and a reference-gas adjustment unit.

In the gas sensor 100 in this embodiment described above in detail, the pump outer electrode 23p and the voltage outer electrode 23s are separately provided outside the sensor element 101. Accordingly, the pump currents Ip0 to Ip3 do not flow through the voltage outer electrode 23s, thus the voltage Vref of the Vref detection sensor cell 83 does not include a voltage drop of the voltage outer electrode 23s due to the pump currents Ip0 to Ip3. Consequently, the voltage Vref has a value which corresponds with higher accuracy to the oxygen concentration in the measurement-object gas outside the sensor element 101, thus the accuracy of detection of the oxygen concentration in the measurement-object gas using the Vref detection sensor cell 83 is improved.

In addition, the sensor element 101 includes a reference-gas adjustment pump cell 90 that has the pump outer electrode 23p and the reference electrode 42, and is configured to pump oxygen from the periphery of the pump outer electrode 23p into the periphery of the reference electrode 42. In this manner, the reference-gas adjustment pump cell 90 pumps oxygen into the periphery of the reference electrode 42, thus reduction in the oxygen concentration of the reference gas in the periphery of the reference electrode 42 can be supplemented.

Furthermore, the controller 96 causes the main pump cell 21 to pump out oxygen from the first internal cavity 20 or pump oxygen into the first internal cavity 20 by controlling the main pump cell 21 so that the oxygen concentration reaches a predetermined low concentration. In this case, the direction of the pump current Ip0 which flows through the main pump cell 21 may be switched to the reverse direction. However, since the pump outer electrode 23p and the voltage outer electrode 23s are separately provided in the sensor element 101, the voltage Vref is not affected by the time required for change in the pump current Ip0. Consequently, when the oxygen concentration in the measurement-object gas is switched between a high state in which the oxygen concentration is higher than a predetermined low concentration and a low state, the responsiveness of the voltage Vref is not likely to reduce.

The present invention is not limited whatsoever to the above embodiments, and various embodiments are possible so long as they belong within the technical scope of the present invention.

For example, in the embodiment described above, the pump outer electrode 23p and the voltage outer electrode 23s are disposed side by side in the front-rear direction, however, may be disposed side by side in the left-right direction. Note that it is preferable that the pump outer electrode 23p and the voltage outer electrode 23s be disposed with a certain gap therebetween so that the voltage Vref does not change due to the effect of the oxygen pumped out into the periphery of the pump outer electrode 23p.

In the above-described embodiment, the fourth diffusion control section 60 is formed as a slit-shaped gap, but is not limited thereto. The fourth diffusion control section 60 may be formed as a porous body (e.g., a ceramic porous body such as alumina (Al₂O₃)). For example, the fourth diffusion control section 60 formed as a porous body may cover the measurement electrode 44. In this case, the periphery of the measurement electrode 44 functions as a measurement chamber. In other words, the periphery of the measurement electrode 44 provides the same function as the third internal cavity 61.

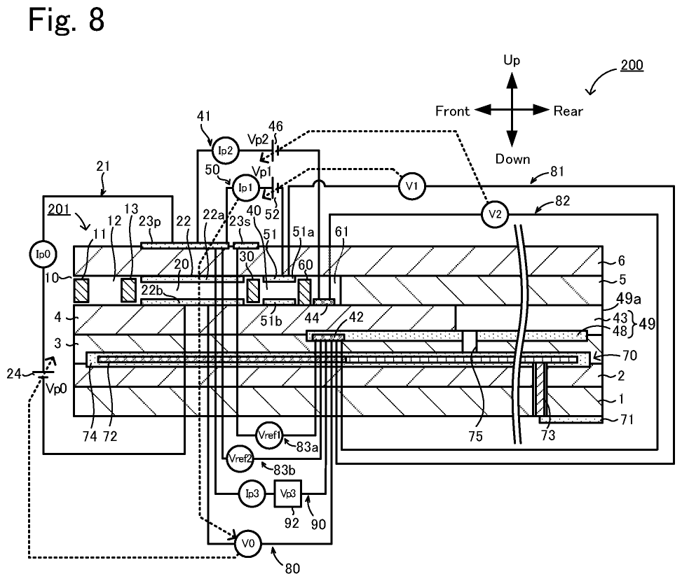

In the above-described embodiment, the controller 96 may obtain not only the voltage Vref across the voltage outer electrode 23s and the reference electrode 42, but also the voltage across the pump outer electrode 23p and the reference electrode 42. FIG. 8 is a schematic cross-sectional view of a gas sensor 200 according to a modification. A sensor element 201 of the gas sensor 200 includes a Vref1 detection sensor cell 83a and a Vref2 detection sensor cell 83b. The Vref1 detection sensor cell 83a is the same sensor cell as the Vref detection sensor cell 83 of the sensor element 101. In the Vref1 detection sensor cell 83a, a voltage Vref1 is generated between the voltage outer electrode 23s and the reference electrode 42. The Vref2 detection sensor cell 83b is an electrochemical sensor cell including: the second solid electrolyte layer 6, the spacer layer 5, the first solid electrolyte layer 4, the third substrate layer 3, the pump outer electrode 23p, and the reference electrode 42. In the Vref2 detection sensor cell 83b, a voltage Vref2 is generated between the pump outer electrode 23p and the reference electrode 42. The gas sensor 200 can determine whether the pump outer electrode 23p is deteriorated based on the difference between the voltage Vref1 and the voltage Vref2. For example, the controller 96 obtains a current Ip4 (e.g., the total value of the pump currents Ip0 to Ip3) which flows through the pump outer electrode 23p, the voltage Vref1 and the voltage Vref2 at a predetermined deterioration determination timing, and calculates the difference Da between the voltage Vref1 and the voltage Vref2 obtained. Next, the controller 96 calculates a reference value for the difference between the voltage Vref1 and the voltage Vref2 based on the obtained current Ip4. The reference value is a value corresponding to the difference between the voltage Vref1 and the voltage Vref2 in a state where the pump outer electrode 23p is not deteriorated. The difference between the voltage Vref1 and the voltage Vref2 includes a voltage drop in the pump outer electrode 23p due to the current which flows through the pump outer electrode 23p, and the controller 96 calculates a reference value based on the obtained pump current Ip4. For example, a relational expression (e.g., the expression of a linear function) and a map representing a correspondence relationship between the current Ip4 and the reference value are pre-stored in the storage unit 98, and the controller 96 calculates a reference value using the obtained current Ip4 and the correspondence relationship. Note that when the rate of the current Ip0 to the current Ip4 (the total value of the currents Ip0 to Ip3) is high, a reference value may be calculated based on the current Ip0 rather than the current Ip4. It is determined whether the pump outer electrode 23p is deteriorated based on whether the difference Da deviates from the reference value (e.g., whether the difference between the difference Da and the reference value exceeds a predetermined threshold). The pump currents Ip0 to Ip3 flow through the pump outer electrode 23p with use of the sensor element 201, thus the pump outer electrode 23p deteriorates. Thus, even when the current which flows through the pump outer electrode 23p is in the same state as before the deterioration, the voltage drop in the pump outer electrode 23p due to the current flow is increased than before the deterioration. Thus, the difference Da between the voltage Vref1 and the voltage Vref2 tends to increase as the pump outer electrode 23p deteriorates. Therefore, the controller 96 can determine whether the pump outer electrode 23p is deteriorated by comparing the difference Da with the aforementioned reference value. When the pump outer electrode 23p deteriorates, the accuracy of measurement of the NOx concentration may be reduced by a change in the values of the pump currents Ip0 to Ip3 which are caused to flow by respective voltages Vp0 to Vp3. When the controller 96 is able to determine deterioration of the pump outer electrode 23p, for example, the controller 96 can prevent the accuracy of measurement of the NOx concentration from remaining at a low level through handling such as transmission of error information to an engine ECU. Note that the controller 96 can determine not only whether the pump outer electrode 23p is deteriorated, but also the degree of deterioration of the pump outer electrode 23p based on the magnitude of the difference Da, or based on the degree of deviation (e.g., the magnitude of the difference between the difference Da and the reference value) between the difference Da and the reference value. In addition, the controller 96 may change control of the sensor element 201 so that effect of deterioration is canceled according to presence or absence of deterioration or the degree of deterioration of the pump outer electrode 23p. For example, the controller 96 may change at least one of the aforementioned target values V0*, V1*, V2*, and Ip1* based on the difference Da or based on the difference between the difference Da and the reference value. Alternatively, the controller 96 may change the amount of oxygen pumped into the periphery of reference electrode 42 by changing the voltage Vp3 to change the pump current Ip3 based on the difference Da or based on the difference between the difference Da and the reference value.

In the above-described embodiment, the sensor element 101 may not include the reference-gas adjustment pump cell 90, and the controller 96 may not include the power supply circuit 92, so that pumping of oxygen into the periphery of the reference electrode 42 by the reference-gas adjustment pump cell 90 may not be provided. Note that when the reference-gas adjustment pump cell 90 pumps oxygen into the reference-gas introduction portion 49, not only the pump currents Ip0 to Ip2 but also the pump current Ip3 flows, thus an electrode (e.g., the outer pump electrode 923 in FIG. 9) through which these pump currents Ip0 to Ip3 are passed is more likely to deteriorate, as compared to when the pump current Ip3 is not passed. Thus, when the reference-gas adjustment pump cell 90 pumps in oxygen, high significance is given to prevention of deterioration of the responsiveness of the voltage Vref by separately providing the pump outer electrode 23p and the voltage outer electrode 23s as in the above-described embodiment.

In the above-described embodiment, the reference-gas adjustment pump cell 90 pumps oxygen from the periphery of the pump outer electrode 23p into the periphery of the reference electrode 42, however, may pump out oxygen from the periphery of the reference electrode 42.

In the above-described embodiment, the element body of the sensor element 101 is a layered body having a plurality of solid electrolyte layers (layers 1 to 6), but is not limited thereto. The element body of the sensor element 101 may include at least one oxygen-ion-conductive solid electrolyte layer, and may be internally provided with a measurement-object gas flow portion. For example, in FIG. 1, the layers 1 to 5 other than the second solid electrolyte layer 6 may be structural layers (e.g., layers composed of alumina) composed of a material other than that of solid electrolyte layers. In this case, the electrodes possessed by the sensor element 101 may be disposed in the second solid electrolyte layer 6. For example, the measurement electrode 44 in FIG. 1 may be disposed on the lower surface of the second solid electrolyte layer 6. Also, the reference-gas introduction space 43 may be provided in the spacer layer 5 instead of the first solid electrolyte layer 4, the reference-gas introduction layer 48 may be provided between the second solid electrolyte layer 6 and the spacer layer 5 instead of between the first solid electrolyte layer 4 and the third substrate layer 3, and the reference electrode 42 may be provided rearward of the third internal cavity 61 and on the lower surface of the second solid electrolyte layer 6.

In the above-described embodiment, the controller 96 sets (feedback-controls) the target value V0* of the voltage V0 based on the pump current Ip1 so that the pump current Ip1 reaches the target value Ip1*, and the controller 96 feedback-controls the voltage Vp0 so that the voltage V0 reaches the target value V0*, but may perform another control. For example, the controller 96 may feedback-control the voltage Vp0 based on the pump current Ip1 so that the pump current Ip1 reaches the target value Ip1*. In other words, the controller 96 may directly control the voltage Vp0 (eventually control the pump current Ip0) based on the pump current Ip1 without obtaining the voltage V0 from the V0 detection sensor cell 80 and setting the target value V0*. Also, in this situation, the controller 96 feedback-controls the voltage Vp1 so that the voltage V1 reaches the target value V1*, thus the controller 96 controls the oxygen concentration in the first internal cavity 20 upstream of the second internal cavity 40 at a predetermined low concentration using the main pump cell 21 so that the pump current Ip1 reaches the target value Ip1* and the oxygen concentration in the second internal cavity 40 reaches a predetermined low concentration (an oxygen concentration corresponding to the voltage V1). Therefore, even when control according to such a modification is performed, as in the description of the embodiment described above, when the oxygen concentration in the measurement-object gas is switched between a high state in which the oxygen concentration is higher than a predetermined low concentration and a low state, the direction of the pump current Ip0 is switched to the reverse direction. Thus, even when control according to such a modification is performed, the effect of preventing reduced responsiveness of the voltage Vref is obtained as in the embodiment described above by separately providing the pump outer electrode 23p and the voltage outer electrode 23s as in the embodiment.

In the above-described embodiment, the oxygen concentration adjustment chamber has the first internal cavity 20 and the second internal cavity 40, however, without being limited to this, for example, the oxygen concentration adjustment chamber may include a still another internal cavity, or one of the first internal cavity 20 and the second internal cavity 40 may be omitted. Similarly, in the above-described embodiment, the adjustment pump cell has the main pump cell 21 and the auxiliary pump cell 50, however, without being limited to this, for example, the adjustment pump cell may include a still another pump cell, and one of the main pump cell 21 and the auxiliary pump cell 50 may be omitted. For example, when the oxygen concentration in the measurement-object gas can be sufficiently reduced to a low oxygen concentration only by the main pump cell 21, the auxiliary pump cell 50 may be omitted. When the auxiliary pump cell 50 is omitted, the controller 96 may omit the aforementioned setting of the target value V0* based on the pump current Ip1.

Specifically, a predetermined target value V0* is pre-stored in the storage unit 98, and the controller 96 may control the main pump cell 21 by feedback-controlling the voltage Vp0 of the variable power supply 24 so that the voltage V0 reaches the target value V0*.

In the above-described embodiment, the gas sensor 100 detects the NOx concentration as a specific gas concentration, however, without being limited to this, another oxide concentration may be used as a specific gas concentration. In the case where the specific gas is an oxide, when the specific gas itself is reduced in the third internal cavity 61, oxygen is produced as in the above-described embodiment, thus the controller 96 can detect a specific gas concentration based on the detection value according to the oxygen. Alternatively, the specific gas may be a non-oxide such as ammonia. In the case where the specific gas is a non-oxide, when the specific gas is converted to an oxide (e.g., ammonia is oxidized and converted to NO), for example, in the first internal cavity and the converted oxide is reduced in the third internal cavity 61, oxygen is produced, thus the controller 96 can obtain a detection value according to the oxygen and detect a specific gas concentration. In this manner, regardless of whether the specific gas is an oxide or a non-oxide, the gas sensor 100 can detect a specific gas concentration based on the oxygen produced from the specific gas in the third internal cavity 61.

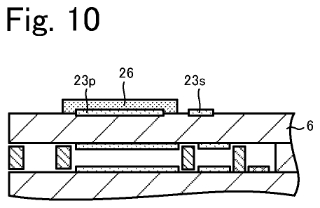

In the above-described embodiment, the pump outer electrode 23p and the voltage outer electrode 23s are exposed to the outside of the sensor element 101, but is not limited thereto. For example, as illustrated in FIG. 10, the pump outer electrode 23p may be covered by a diffusion layer 26. The diffusion layer 26 is disposed on the upper surface of the second solid electrolyte layer 6 to cover the entire pump outer electrode 23p. The diffusion layer 26 does not cover the voltage outer electrode 23s, which is exposed to the outside of the sensor element 101. The diffusion layer 26 is formed as a porous body (e.g., a ceramic porous body such as alumina (Al₂O₃)), and adds a diffusion resistance to the measurement-object gas which reaches the pump outer electrode 23p from the outside of the sensor element 101. Decrease in the accuracy of measurement of the NOx concentration in the measurement-object gas can be prevented by covering the pump outer electrode 23p with the diffusion layer 26. The reason for this is probably as follows. First, as described above, the pump outer electrode 23p contains a noble metal having catalytic activity, thus reduction of NOx in the measurement-object gas may occur in the periphery of the pump outer electrode 23p. Particularly, when the measurement-object gas is in a rich atmosphere (including when in a slightly rich atmosphere) or has a theoretical air-fuel ratio (stoichiometric ratio), the controller 96 controls the main pump cell 21 to pump oxygen into the first internal cavity 20, thus the oxygen concentration in the periphery of the pump outer electrode 23p is decreased to produce a reducing atmosphere, and NOx reduction is likely to occur in the periphery of the pump outer electrode 23p. When NOx reduction occurs in the periphery of the pump outer electrode 23p, the measurement-object gas with a decreased NOx concentration is introduced into the measurement-object gas flow portion of the sensor element 101 through the gas inlet 10, and the accuracy of measurement of the NOx concentration may be decreased. At this point, when the pump outer electrode 23p is covered by the diffusion layer 26, it is difficult for the measurement-object gas to reach the periphery of the pump outer electrode 23p, thus the amount of NOx reduced per unit time in the periphery of the pump outer electrode 23p is decreased. In addition, since the pump outer electrode 23p is covered by the diffusion layer 26, the amount of the measurement-object gas which follows a path from arrival to the periphery of the outer pump electrode 23 to the gas inlet 10 is decreased. As a result, the aforementioned decrease in the accuracy of measurement of the NOx concentration due to NOx reduction in the periphery of the pump outer electrode 23p is prevented. In contrast, since the voltage outer electrode 23s is not covered by the diffusion layer 26, the measurement-object gas outside the sensor element 101 is more likely to reach the pump outer electrode 23p, as compared to when the voltage outer electrode 23s is covered by the diffusion layer 26. Therefore, reduction in the responsiveness of the voltage Vref is prevented, as compared to when the voltage outer electrode 23s is covered by the diffusion layer 26. As described above, the voltage outer electrode 23s is smaller in area than the pump outer electrode 23p, no current flows through the voltage outer electrode 23s, and no oxygen is pumped out from the periphery of the voltage outer electrode 23s into the measurement-object gas flow portion, thus NOx reduction is unlikely to occur in the periphery of the voltage outer electrode 23s. Thus, even if the voltage outer electrode 23s is not covered by the diffusion layer 26, NOx reduction is unlikely to occur.

In the above-described embodiment, the pump outer electrode 23p and the voltage outer electrode 23s each contain a noble metal having catalytic activity, however, the pump outer electrode 23p may further contain a noble metal (e.g., Au) having a catalytic activity inhibition ability to inhibit the catalytic activity. NOx reduction can be prevented from occurring in the periphery of the pump outer electrode 23p by containing a noble metal having a catalytic activity inhibition ability in the pump outer electrode 23p, thus the aforementioned decrease in the accuracy of measurement of the NOx concentration due to the NOx reduction is prevented. Although a noble metal having a catalytic activity inhibition ability may be contained in the voltage outer electrode 23s, it is preferable that no noble metal having a catalytic activity inhibition ability be contained in the voltage outer electrode 23s because in that case, decrease in the responsiveness of the voltage Vref is prevented. The inventors verified this by experiments and analysis. A noble metal having a catalytic activity inhibition ability may be contained in the pump outer electrode 23p, and the pump outer electrode 23p may be covered by the diffusion layer 26 illustrated in FIG. 10.

What is claimed is:

1. A sensor element for detecting a specific gas concentration in a measurement-object gas, the sensor element comprising:
an element body including an oxygen-ion-conductive solid electrolyte layer and internally provided with a measurement-object gas flow portion that introduces a measurement-object gas and causes the measurement-object gas to flow therethrough;
an adjustment chamber pump cell having an adjustment electrode disposed in an oxygen concentration adjustment chamber of the measurement-object gas flow portion, and a pump outer electrode disposed outside the element body, the adjustment chamber pump cell being configured to pump out oxygen from the oxygen concentration adjustment chamber or pump oxygen into the oxygen concentration adjustment chamber;
a measurement pump cell having a measurement electrode disposed in a measurement chamber provided downstream of the oxygen concentration adjustment chamber of the measurement-object gas flow portion, and the pump outer electrode, the measurement pump cell being configured to pump out oxygen produced from the specific gas in the measurement chamber;
a reference-gas introduction portion disposed inside the element body, the reference-gas introduction portion being configured to introduce a reference gas serving as a reference for detecting a specific gas concentration in the measurement-object gas; and
an outer sensor cell having a voltage outer electrode disposed outside the element body, and a reference electrode disposed inside the element body so as to be in contact with the reference gas introduced to the reference-gas introduction portion, the outer sensor cell being configured to generate a voltage based on an oxygen concentration in the measurement-object gas outside the element body.

2. The sensor element according to claim 1, further comprising:
a reference-gas adjustment pump cell having the pump outer electrode and the reference electrode, the reference-gas adjustment pump cell being configured to pump oxygen into a periphery of the reference electrode from a periphery of the pump outer electrode.

3. A gas sensor comprising:
the sensor element according to claim 1;
an adjustment chamber pump cell controller that causes the adjustment chamber pump cell to pump out oxygen from the oxygen concentration adjustment chamber or pump oxygen into the oxygen concentration adjustment chamber by controlling the adjustment chamber pump cell so that an oxygen concentration in the oxygen concentration adjustment chamber reaches a predetermined low concentration; and
an oxygen concentration detector that detects an oxygen concentration in the measurement-object gas outside the element body based on the voltage of the outer sensor cell.

4. A gas sensor comprising:
the sensor element according to claim 2;
an adjustment chamber pump cell controller that causes the adjustment chamber pump cell to pump out oxygen from the oxygen concentration adjustment chamber or pump oxygen into the oxygen concentration adjustment chamber by controlling the adjustment chamber pump cell so that an oxygen concentration in the oxygen concentration adjustment chamber reaches a predetermined low concentration; and
an oxygen concentration detector that detects an oxygen concentration in the measurement-object gas outside the element body based on the voltage of the outer sensor cell.
